(19) United States
(12) Patent Application Publication
(10) Pub. No.: US 2015/0182673 A1
(43) Pub. Date: Jul. 2, 2015

(54) FUNCTIONALIZED LUBRICIOUS MEDICAL DEVICE COATINGS

(71) Applicant: Boston Scientific Scimed, Inc., Maple Grove, MN (US)

(72) Inventors: Joseph Thomas Delaney, JR., Minneapolis, MN (US); Kasyap Seethamraju, Eden Prairie, MN (US); John Kummailil, Sherborn, MA (US); Paul Vincent Grosso, Maple Grove, MN (US)

(21) Appl. No.: 14/581,404

(22) Filed: Dec. 23, 2014

Related U.S. Application Data
(60) Provisional application No. 61/921,646, filed on Dec. 30, 2013.

(51) Int. Cl. A61L 31/14; A61L 27/50; A61L 29/14; A61L 27/34; A61L 31/10; A61L 29/08

(52) U.S. Cl. CPC A61L 31/14; A61L 31/10; A61L 29/085; A61L 29/14; A61L 27/34; A61L 27/50; A61L 2400/10

(57) ABSTRACT

In some aspects of the present invention, coatings are provided which provide lubricity as well as additional functionality. Further aspects of the invention pertain to medical devices having such coatings and methods of forming such coatings.

### CLAIM OF PRIORITY

This application claims priority to U.S. Provisional Application Ser. No. 61/921,646, filed on Dec. 30, 2013, the entire contents of which are hereby incorporated by reference.

### BACKGROUND

The therapeutic utility of medical devices often benefits from the use of hydrophilic coatings. Such coatings often employ a hydrogel chemistry to impart a soft, low-friction surface. Oftentimes, the durability of this coating can be improved by covalently bonding the material directly to the surface, as is done, for example, with commercial products such as Polyslip™, among others. See, e.g., You-ling Fan, EP0379156A2 and C. Rouns, et al. U.S. Pat. No. 7,220,491. It would be highly desirable, however, to provide hydrophilic coatings that have additional functionality beyond lubricity.

### BRIEF SUMMARY

In some aspects of the present invention, coatings are provided which provide lubricity as well as additional functionality. Further aspects of the invention pertain to medical devices having such coatings and methods of forming such coatings.

For example, in some embodiments, the present disclosure is directed to medical devices that comprise a substrate material and a lubricious coating disposed on the substrate material, wherein the coating comprises (a) a hydrophilic polymer species, (b) a functional species and (c) a coupling species.

As another example, in some embodiments, the present disclosure is directed to methods for forming medical device coatings that comprise (a) applying a first coating comprising a coupling species to a substrate material and (b) applying to the first coating one or more additional coatings that comprise a hydrophilic polymer species and a functional species.

These and other aspects and embodiments as well as various advantages of the present invention will become immediately apparent to those of ordinary skill in the art upon review of the Detailed Description and claims to follow.

### BRIEF DESCRIPTION OF FIGURES

FIG. 1 is a schematic illustration of a method of forming a device coating, in accordance with an embodiment of the present invention.

### DETAILED DESCRIPTION

In the present disclosure, crosslinked hydrophilic polymers are employed to impart a low-friction surface to medical devices. In certain embodiments, the durability of this coating can be improved by covalently bonding the coating to the medical device surface.

While lubricity can be an important characteristic of a medical device coating, in the present disclosure, additional properties are provided to the coating through the addition of one or more functional species that provide, for instance, therapeutic agent delivery, diagnostic functionality and/or cellular adhesion properties, among others.

Coatings in accordance with the present disclosure comprise (a) at least one hydrophilic polymer species, (b) at least one functional species and (c) at least one coupling species. As discussed in more detail below, such coatings can be formed on a variety of substrate materials from a wide range of hydrophilic polymer species, functional species and coupling species.

### Coupling Species

Coupling species for use in conjunction with the present disclosure may, for example, perform one or more of the following functions: couple the hydrophilic polymer species to the underlying substrate, couple the hydrophilic polymer species to one another, couple the hydrophilic polymer species to the functional species, couple the functional species to the underlying substrate and couple the functional species to one another.

In various embodiments, coupling species are polyfunctional (e.g., difunctional, trifunctional, tetrafunctional, etc.) in nature, and are capable of reacting with functional groups present in the substrate material, functional groups present in the hydrophilic polymer species, functional groups present in the functional species, or in a combination of two or more the foregoing. These coupling reactions may result in the formation of a variety of covalent bond-based linking functional groups, including for example, ester groups (—CO—O—), thioester groups (—CO—S—), thioether groups (—S—), anhydride groups (—CO—O—CO—), amide groups (—NH—CO—), urethane groups (—NH—CO—O—), thiol-urethane groups (—NH—CO—S—), urea groups (—NH—CO— NH—), silicon-based groups (e.g., ≡Si—O— groups, ≡Si—O—CO— groups, ≡Si—N— groups, ≡Si—S— groups, etc.), and acetal groups (—O—CHR—O—), among others. Other linking chemistries involving more complex structures affording a covalent link between the substrate and the functional species are also possible, such as through thiol-ene addition chemistry yielding thioethylene linking group (—S—CH₂—CH₂—), Michael-type addition through a sulfonylethylene group (—SO₂—CH₂—CH₂—), through a Huisgen 1,3-dipolar cycloaddition (bonding through the creation of a 1,2,3-triazole ring species), through a Diels-Alder [4+2] cycloaddition, through biotinylation, through [4+1] cycloadditions between isonitriles and tetrazines (diazoles), and through small ring opening addition reactions of oxetanes or epoxides reacting with nucleophiles, among others.

In certain beneficial embodiments, polyfunctional isocyanates (also referred to as polyisocyanates) may act as coupling species. For example, the isocyanate groups of the polyisocyantes can be reacted with alcohol groups to form urethane bonds, with thiol groups to form thiol-urethane bonds, and with amine groups to form urea bonds, among other possibilities.

A variety of polyisocyanates can be employed in preparing the coatings of the present disclosure. Specific examples of polyisocyanates include aliphatic and aromatic diisocyates such as methylene diphenyl diisocyanate (MDI), polymeric MDI, toluene diisocyanate (TDI), hexamethylene diisocyanate (HDI), hydrogenerated diphenylmethane diisocyanate (12H-MDI) and isophorone diisocyanate (IPDI), among others.

Further specific examples of polyisocyanates include isocyanate-terminated prepolymers including, for example, reaction products of (1) a diisocyanate such as MDI, TDI, 12H-MDI or HDI, among others with (2) a diol. The diol may be selected from one or more of the following, among others: (a) ethane diols, which may be present as one or more isomers such as 1,2-ethane diol (also known as ethylene glycol) and polyethane diols (e.g., polyethylene glycols such as diethylene glycol, triethylene glycol, tetraethylene glycol, etc.), (b) propane diols, which may be present as one or more isomers such as 1,2-propane diol (also known as propylene glycol) and 1,3-propane diol (also known as trimethylene glycol or 1,3 propylene glycol), and polypropane diols (e.g., polypropylene glycols such as dipropylene glycol, tripropylene glycol, tetrapropylene glycol, etc.), (c) butane diols, which may be present as one or more isomers such as 1,2-butane diol (also known as 1,2-butylene glycol or alpha-butylene glycol), 1,3-butane diol (also known as 1,3-butylene glycol), 1,4-butane diol (also known as 1,4-butylene glycol or tetramethylene glycol), and 2,3-butane diol (also known as 2,3-butylene glycol), and polybutane diols (e.g., polybutylene glycols such as dibutylene glycol, tributylene glycol, tetrabutylene glycol, etc.) and (d) higher alkane diols, and higher polyalkane and polyether diols.

While polyisocyanates are used as exemplary polyfunctional coupling species herein, other coupling species are suitable for use in conjunction with the present disclosure, including polyfunctional acyl chlorides, polyfunctional silane compounds, and polyfunctional amines in the presence of carbodiimides and/or “BOP” coupling reagents, among others. In this regard, polyfunctional acyl chlorides can act as coupling species by reaction, for example, with alcohol functional groups (thereby forming ester bonds), with carboxyl functional groups (thereby forming acid anhydride bonds), with thiol functional groups (thereby forming thioester bonds) and/or with amine functional groups (thereby forming amide bonds), among others. Polyfunctional silanes (e.g., chloro-silanes, alkoxy-silanes, acetoxy-silanes, etc.) can act as coupling species by reaction, for example with alcohol functional groups (thereby forming ≡Si—O— bonds), with carboxyl functional groups (thereby forming ≡Si—O—CO— bonds), with amine functional groups (thereby forming ≡Si—NH— bonds) and/or with thiol functional groups (thereby forming ≡Si—S— bonds), among others.

### Hydrophilic Polymer Species

Hydrophilic polymer species may be selected, for example, from homopolymers and copolymers containing one or more of the following monomers, among others: hydrophilic acrylic monomers such as acrylic acid and salts thereof as well as hydroxyalkyl acrylates, including hydroxyethyl acrylate, hydrophilic methacrylic monomers such as methacrylic acid and salts thereof as well as hydroxyalkyl methacrylates including hydroxyethyl methacrylate, polyethylene glycol methacrylate, oligoethylene glycol methacrylate, other acidic monomers such as vinyl sulfonic acid and salts thereof, amine-based monomers such as vinyl amine, allylamine, vinyl pyrrolidone and alkyleneimines such as ethyleneimine (e.g., aziridine), amide based monomers such as acrylamide, hydroxy-olefin monomers such as vinyl alcohol, zwitterionic monomers, including sulfobetaine monomers such as sulfobetaine methacrylate, sulfobetaine acrylate, and sulfobetaine acrylamide monomers, ether monomers such as methyl vinyl ether and alkyl ether monomers such as ethylene oxide, anhydrides such as maleic anhydride, saccharides, and amino acids such as lysine, arginine, histidine, aspartic acid and glutamic acid.

Hydrophilic polymer species may be selected, for example, from biopolymers including proteins such as collagen or gelatin, or polysaccharides, such chitin, chitosan, starch, carboxymethyl starch, as well as other starches, inulin, cellulosic polymers such as carboxymethyl cellulose, dextran, dextrin, carboxymethyl dextran, modified dextran, alginic acid, pectinic acid, hyaluronic acid, chitin, pullulan, gellan, xanthan, chondroitin sulfate, guar, and derivatives and mixtures of the foregoing.

In certain embodiments, hydrophilic polymers are selected which have reactive end-groups, and include, for example, amine-terminated polymers (e.g., diamines, triamines and higher polyamines), hydroxyl-terminated polymers (e.g., diols, triols and higher polyols), thiol-terminated polymers (e.g., dithiols, trithiols and higher polythiols).

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. Polymers for use in the present disclosure can be linear or branched. Branched configurations include star-shaped configurations (e.g., configurations in which three or more chains emanate from a single branch point), comb configurations (e.g., configurations having a main chain and a plurality of side chains), dendritic configurations (e.g., arborescent and hyperbranched polymers), and so forth.

### Functional Species

In various aspects of the present disclosure, coatings are provided which contain one or more functional species, examples of which include species that have a therapeutic effect, species that have a diagnostic capability and species affecting adhesion of cellular and non-cellular species, among others.

Functional species may be associated with the devices of the present disclosure via various mechanisms. For example, in some embodiments, functional species may be associated with the devices (e.g., associated with a substrate surface, associated with hydrophilic polymer species, associated with particles, etc.) through non-covalent interactions such as physical entrapment, van der Waals forces, hydrophobic interactions and/or electrostatic interactions (e.g., charge-charge interactions, charge-dipole interactions, and dipole-dipole interactions, including hydrogen bonding). In some embodiments, functional species may be associated with the devices by covalent bonds, for example, bound via a suitable coupling species (e.g., a polyisocyanate, polyfunctional acyl chloride, polyfunctional silane compound, etc.) through functional groups (e.g., hydroxyl, thiol, amine, carboxyl, etc. groups) found on the functional species and functional groups found elsewhere in the device (e.g., functional groups found on the substrate surface, on the hydrophilic polymers chains of the coating, on particles provided in the coating, etc.).

As previously indicated, in some embodiments, functional species in accordance with the present disclosure may be associated with a particle. For example, in some embodiments, the functional species itself may be in the form of a particle. In some embodiments, the functional species may be coupled to the surface of particulate carrier, for example, through functional groups found on the particle surface. In some embodiments, the functional species may be located within a particulate carrier, for example, by blending the functional species with a particulate carrier material such that the functional species is dispersed throughout the particulate carrier material or by encapsulating the functional species within a particulate carrier material. In such cases, the particulate carrier may be designed to release the functional species from the device over time (e.g., by diffusion, particle degradation, etc.) or to retain the functional species in association with the device.

“Particle size” is defined herein as the smallest of the particle's three dimensions (e.g., the diameter of a spherical particle, the width of a fiber, the thickness of a plate-shaped particle, etc.). In some embodiments, functional species in accordance with the present disclosure may be associated with a nanoparticle. “Nanoparticles” are defined herein as particles having a particle size that is less than 1 μm (1000 nm), for example, a particle size ranging from 1 nm to 2.5 nm to 5 nm to 10 nm to 25 nm to 50 nm to 100 nm to 250 nm to 500 nm to 1000 nm). In many embodiments, two or all three of the nanoparticle's dimensions are less than 1 μm.

Particle carrier materials include organic and inorganic carrier materials, for example comprising one or more polymers selected from the hydrophilic polymers set forth herein for use as hydrophilic polymer species. Particle carrier materials may also be selected, for example, from one or more suitable inorganic and/or organic materials listed below in conjunction with substrate materials, among others.

### Therapeutic Functional Species

In some aspects of the present disclosure, coatings are provided with therapeutic functional species. As defined herein, a “therapeutic functional species” or “therapeutic species” is a species that is administered to a patient for use in the treatment, cure, detection or prevention of a disease or condition.

Therapeutic functional species may be associated with the device in various ways, including those discussed above, for example, by non-covalent interactions with the substrate surface, by covalent bonding with the substrate surface, by non-covalent interactions with the hydrophilic polymer species forming the coating, by covalent bonding with the hydrophilic polymer species forming the coating, or by association with a particle.

In some embodiments, the therapeutic functional species is released from the device upon administration to a patient (e.g. as a result of reversible non-covalent binding, as a result of degradation of covalent bonds, as a result of particle degradation, etc.). In some embodiments, the therapeutic functional species remains bound to the device.

A variety of therapeutic functional species may be employed in the present disclosure including gene vectors (e.g., plasmids, viral vectors, cosmids, artificial chromosomes, etc.), adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anti-cancer supplementary potentiating agents, antineutropenic agents, antiobsessional agents, antiparasitic agents, antiparkinsonian drugs, antipneumocystic agents, antiproliferative agents, antiprostatic hypertrophy drugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine A1 antagonists, serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, and xanthine oxidase inhibitors, among others.

In specific embodiments, the therapeutic functional species is a species having antimicrobial activity. Examples of antimicrobial agents for use in the present disclosure may be selected, for example, from triclosan, chlorhexidine, nitrofurazone, benzalkonium chlorides, silver salts, silver particles, metallic silver and antibiotics, such as rifampin, gentamicin and minocycline, and combinations thereof, among others.

In accordance with one specific embodiment, silver nanoparticles are employed to inhibit the viability of bacteria on the device surface. (For further information on silver nanoparticles, see, e.g., US Patent Pub. 2002/0182265A1 and WO2008031601A1.) Metal nanoparticles, including silver and gold nanoparticles, may prepared by reduction of salts, with polymer additives that are selected to control their size distribution, growth, geometry, and colloid stability. In certain cases, the polymers selected contain highly polar groups (e.g., polyvinyl pyrrolidone, polyvinyl alcohol, etc.), resulting in “capped” colloidal dispersions (e.g., dispersions in which a “capping agent” acts to cease reduction of the metal salts and thus particle growth). For example, the preparation of polyvinyl alcohol (PVA) capped silver nanoparticles has been reported. R. S. Patil et al., “One-pot synthesis of PVA-capped silver nanoparticles their characterization and biomedical application,” Advances in Natural Sciences: Nanoscience and Nanotechnology, 3 (2012) 015013 (7pp). The polymer in the metal nanoparticles may be bound to the device by various mechanisms (e.g., non-covalent or covalent bonding to the substrate surface, to hydrophilic polymer species forming the coating, etc.). In a specific example, the PVA in a metallic nanoparticle (e.g., a nanoparticle containing silver, gold, etc.) is linked to a hydrophilic polymer species in the coating (e.g., PVA, hydroxy terminated polyacrylic acid, etc.) by using a diisocyanate to form stable urethane linkages between the nanoparticle and the hydrophilic polymer species.

In other specific embodiments, the therapeutic functional species is a species having analgesic and/or anti-inflammatory characteristics, for example, selected from narcotic analgesic agents, non-narcotic analgesic agents, local anesthetic agents, anti-inflammatory steroid drugs and nonsteroidal anti-inflammatory drugs, and combinations thereof.

For instance, medical devices such as guidewires and catheters are frequently manipulated through tortuous anatomical features, potentially causing pain and/or inflammation during the process. In certain embodiments, devices in accordance with the present disclosure are provided with analgesic and/or anti-inflammatory species to address these conditions, for example, by releasing such species such that they are deposited on and/or absorbed by lumen walls which come into close proximity to (e.g., contact) the device coating, thereby reducing post-operative discomfort and/or inflammation.

### Diagnostic Functional Species

In some aspects of the present disclosure, devices are provided with diagnostic functional species. Diagnostic functional species may be associated with the device in various ways, including those discussed above, for example, by non-covalent interactions with the substrate surface, by covalent bonding with the substrate surface, by non-covalent interactions with the hydrophilic polymer species forming the coating, by covalent bonding with the hydrophilic polymer species forming the coating, or by association with a particle (e.g., the diagnostic functional species may itself be in the form of a particle, the diagnostic functional species may be coupled to a surface of a particulate carrier, the diagnostic functional species may be positioned within a suitable particulate carrier, etc.), which particle may be covalently or non-covalently bound to the device.

In certain of embodiments, coatings in accordance with the present disclosure are provided with diagnostic functional species that interact with an analyte of interest in a body fluid such as urine, blood, gastric juices, lymph, cerebrospinal fluid, synovial fluid, pleural fluid, pericardial fluid, bile, amniotic fluid, peritoneal fluid or feces, among others, allowing the analyte to be detected. As a specific example, a urine contacting device such as a catheter may be provided that detects sugar levels, proteinuria or urinary tract infection products, among other analytes, in urine. As another specific example, a blood-contacting device such as a catheter may be provided that determines HbAlc (Hemoglobin Al c) levels, insulin levels or liver enzyme levels, among other analytes, in blood. As another specific example, a catheter that delivers contrast agent during an interventional cardiology procedure may be provided that determines serum creatinine level, a marker for kidney damage.

In certain embodiments, coatings in accordance with the present disclosure are provided with diagnostic functional species that include one or more components of an enzyme-linked immunosorbent assay. For instance, the presence of analyte species such as protein biomarkers, pathogens, and/or rare cell types may be detected with the aid of an implantable or insertable medical device. For example, a catheter may be used to allow direct and immediate access to a site where the concentration of analyte species are expected to be the highest, giving enhanced sensitivity. The surface of the catheter may be modified, for example, by attaching capture antibodies for the analyte species, thereby allowing the surface of the catheter to act as sampling platform for such species. The analysis could then be done ex vivo or in vivo, as desired. For example, the catheter surface may be exposed to enzyme-containing species that bind to the analyte species, followed by the addition of a substrate for the enzyme, which yields a color change when the analyte species is present in sufficient concentration.

In other embodiments, hydrophilic coatings in accordance with the present disclosure are provided with diagnostic functional species that comprise imaging agents. Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with x-ray fluoroscopy, including metals and metal compounds (e.g., metal salts, metal oxides, etc.), for instance, barium compounds, bismuth compounds and tungsten, among others, and iodinated compounds, among others, (e) radiocontrasting agents, such as those based on the clinically important isotope ^{99m}Tc, as well as other gamma emitters such as ¹²³I, ¹²⁵I, ¹³¹I, ¹¹¹In, ⁵⁷ Co, ¹⁵³Sm, ¹³³Xe, ⁵¹Cr, ^{81m}Kr, ²⁰¹Tl, ⁶⁷ Ga, and ⁷⁵Se, among others, (f) positron emitters, such ¹⁸F, ¹¹C, ¹³N, ¹⁵O, and ⁶⁸Ga, among others, may be employed to yield functionalized radiotracer coatings, and (g) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

### Adhesion Functional Species

In some aspects of the present disclosure, coatings are provided with functional species that affect (i.e., promote or inhibit) adhesion of cells or non-cellular chemical species such as salts, proteins, cytokines, and/or other macromolecules involved in cell anchoring, cell migration, and tissue ingrowth. Such adhesion functional species may, for example, promote adhesion of cells (e.g., progenitor cells, vascular endothelial cells, fibroblasts, macrophages etc.) to the device surface, inhibit adhesion of cells (e.g., microbial cells, foam cells, etc.) to the device surface, promote adhesion of non-cellular chemical species (e.g., therapeutically advantageous interleukins, growth factors, and related cytokines) to the device surface, inhibit adhesion of non-cellular chemical species to the device surface (e.g., plaques, calcium deposits, and other stenotic materials), promote or inhibit adhesion of cells to one another, promote or inhibit adhesion of non-cellular chemical species to one another, or promote or inhibit adhesion of non-cellular chemical species to cells, and so forth.

Such adhesion functional species may be associated with the device in various ways, including those discussed above, for example, by non-covalent interactions with the substrate surface, by covalent bonding with the substrate surface, by non-covalent interactions with the hydrophilic polymer species forming the coating, by covalent bonding with the hydrophilic polymer species forming the coating, or by association with a particle. In some embodiments, the adhesion functional species is released from the device upon administration to a patient (e.g., due to reversible non-covalent binding, degradation of covalent bonds, particle degradation, swelling of a matrix polymer, etc.). In some embodiments, the adhesion functional species remains bound to the device.

Adhesion functional species affecting cellular adhesion may also be selected, for example, from suitable members of the following (or active portions thereof), among others: cell signaling proteins, growth factors, cytokine receptors, interleukins, extracellular materials such as submucosa, bone marrow, extracellular membrane, and basement membrane, various components of extracellular materials, including fibrous materials and ground substance (e.g., glycosaminoglycans, proteoglycans, and glycoproteins), for instance, collagen, laminin, elastin, fibronectin, heparin sulfate, hyaluron, dermatan sulfate, keratin sulfate, and chrondroitin sulfate, among others, adhesive species such as ankyrins, cadherins, members of the immunoglobulin superfamily (which includes a wide array of molecules, including NCAMs, ICAMs, VCAMs, and so forth), selectins (L-, E- and P-subclasses), connexins, immunoglobulins, mucoadhesives, sialyl Lex, plant or bacterial lectins (adhesion molecules which specifically bind to sugar moieties of the epithelial cell membrane), integrins, entactin, fibrin, vimentin, glycolipids, glycophorin, glycoproteins, hyaluronic acid, spektrin, von Willebrand factor, vinculin, vitronectin, and species (including peptides and proteins) that contain cell adhesion peptides such as RGD tripeptide (i.e., ArgGlyAsp, which has been identified to be responsible for some of the cell adhesion properties of fibronectin, laminin, collagen I, collagen IV, thrombospondin, and tenascin), REDV tetrapeptide (i.e., Arg-Glu-Asp-Val), which has been shown to support endothelial cell adhesion but not that of smooth muscle cells, fibroblasts, or platelets, and YIGSR pentapeptide (i.e., TyrIleGlySerArg), which promotes epithelial cell attachment, but not platelet adhesion). More information on RGD, REDV, and YIGSR peptides can be found in U.S. Pat. No. 6,156,572 and U.S. Patent Application No. 2003/0087111. A further example of a bioactive species is NGR tripeptide, which binds to CD13 of endothelial cells. See, e.g., L. Holle et al., “In vitro targeted killing of human endothelial cells by co-incubation of human serum and NGR peptide conjugated human albumin protein bearing alpha (1-3) galactose epitopes,” Oncol. Rep. March 2004; 11(3):613-6.

Adhesion functional species affecting (in particular, inhibiting) cellular adhesion may also include synthetic polymers such as polyethylene glycol and pegylated species (i.e., species with covalently attached polyethylene glycol polymer chains).

Examples of adhesion functional species may further include anithrombogenic species such as heparin (which binds to antithrombin III thereby preventing the formation of clots and extension of existing clots within the blood), anti-calcification agents such as bisphosphonates (which bind to calcium compounds and inhibit calcium oxalate crystal growth), and fibroblast growth factor (FGF) to promote controlled localized proliferation and differentiation of endothelial cells.

### Substrate Materials

Coatings in accordance with the present disclosure can be formed on a wide variety of substrate materials. Substrate materials may be selected, for example, from (a) organic materials such as polymeric materials and biologics, (b) inorganic materials, such as metallic materials and non-metallic materials and (c) hybrid materials (e.g., hybrid organic-inorganic materials, for instance, polymer/metallic hybrids, polymer/ceramic hybrids, etc.). Substrate materials may be biostable or bioerodable.

Specific examples of metallic materials may be selected, for example, from biostable metals such as gold, iron, niobium, platinum, palladium, iridium, osmium, rhodium, titanium, tantalum, tungsten, ruthenium, zinc, and magnesium, among others, biostable alloys such as those comprising iron and chromium (e.g., stainless steels, including platinum-enriched radiopaque stainless steel), niobium alloys, titanium alloys, alloys comprising nickel and titanium (e.g., Nitinol), alloys comprising cobalt and chromium, including alloys that comprise cobalt and chromium (e.g., Elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), bioerodable metals such as magnesium, zinc and iron, and bioerodable alloys including alloys of magnesium, zinc and/or iron (and their alloys with combinations of Ce, Ca, Al, Zr, La and Li), among others.

Specific examples of inorganic non-metallic materials may be selected, for example, from biostable and bioerodable materials containing one or more of the following: nitrides, carbides, borides, and oxides of various metals, including those above, among others, for example, aluminum oxides and transition metal oxides (e.g., oxides of iron, zinc, magnesium, titanium, zirconium, hafnium, tantalum, molybdenum, tungsten, rhenium, niobium, and iridium); silicon; silicon-based ceramics, such as those containing silicon nitrides, silicon carbides and silicon oxides (sometimes referred to as glass ceramics); various metal- and non-metal-phosphates, including calcium phosphate ceramics (e.g., hydroxyapatite); other bioceramics; calcium carbonate; carbon; and carbon-based, ceramic-like materials such as carbon nitrides.

Specific polymers may be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,1-and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-l-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF) poly(vinylidene fluoride-co-hexafluoropropylene) (PVDF-HFP); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as further copolymers and blends of the above.

### Medical Devices

Coatings in accordance with the present disclosure may be applied to a wide variety of medical devices, including implantable or insertable medical devices, which may be selected, for example, from wire interventional devices such as guidewires, diagnostic devices such as pressure wires, catheters including urological catheters and vascular catheters, such as balloon catheters and various central venous catheters, balloons, vascular access ports, dialysis ports, stents (including coronary vascular stents, peripheral vascular stents, cerebral, urethral, ureteral, biliary, tracheal, gastrointestinal and esophageal stents), stent grafts, vascular grafts, abdominal aortic aneurysm (AAA) devices (e.g., AAA stents, AAA grafts, etc.), filters (e.g., vena cava filters and mesh filters for distal protection devices), embolization devices including cerebral aneurysm filler coils (including Guglielmi detachable coils and metal coils), embolic agents, septal defect closure devices, drug depots that are adapted for placement in an artery for treatment of the portion of the artery distal to the device, myocardial plugs, pacemakers, leads including pacemaker leads, defibrillation leads and coils, neurostimulation leads such as spinal cord stimulation leads, deep brain stimulation leads, peripheral nerve stimulation leads, cochlear implant leads and retinal implant leads, ventricular assist devices including left ventricular assist hearts and pumps, total artificial hearts, shunts, valves including heart valves and vascular valves, anastomosis clips and rings, tissue bulking devices, suture anchors, tissue staples and ligating clips at surgical sites, cannulae, metal wire ligatures, tacks for ligament attachment and meniscal repair, joint prostheses, spinal discs and nuclei, orthopedic prosthesis such as bone grafts, bone plates, fins and fusion devices, orthopedic fixation devices such as interference screws in the ankle, knee, and hand areas, rods and pins for fracture fixation, screws and plates for craniomaxillofacial repair, dental implants, or other devices that are implanted or inserted into the body.

### Processing

Coatings may be formed using a number of techniques. In many embodiments, the species are applied to a substrate as a solution and/or suspension in a suitable organic solvent.

In some embodiments, at least one hydrophilic polymer species, at least one functional species and at least one reactive coupling species are applied to a surface simultaneously.

In some embodiments, at least one hydrophilic polymer species and at least one reactive coupling species are applied to a surface simultaneously in a first layer. Then, at least one functional species, either with our without at least one reactive coupling species, is applied as a second layer on top of the first layer. In other embodiments, at least one functional species and at least one reactive coupling species are applied to a surface simultaneously in a first layer. Then, at least one hydrophilic polymer species, either with our without at least one reactive coupling species, is applied as a second layer on top of the first layer.

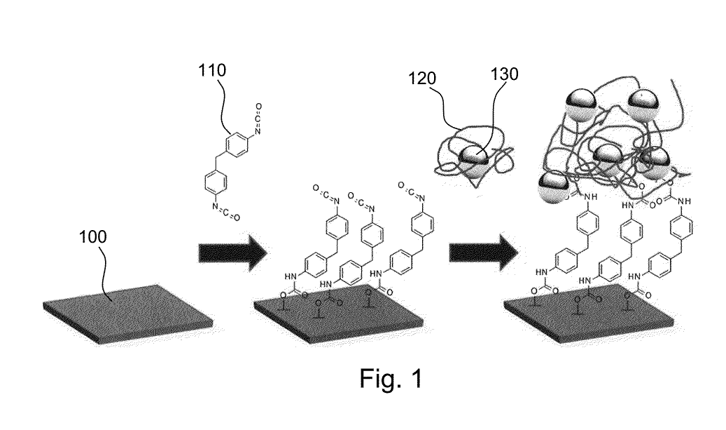

In still other embodiments, at least one coupling species is applied as a first priming coat to a substrate surface, thereby forming a chemically reactive surface. Then one or more hydrophilic polymer species and one or more functional species are applied, either with or without additional coupling species. For example, the one or more hydrophilic polymer species and one or more functional species may be applied simultaneously as a second coat to the first priming coat. An example of such as process can be seen with reference to FIG. 1, which schematically illustrates a process in which a coupling species 110 (e.g., a polyisocyanate such as a diisocyanate, as shown, and/or an isocyanate-terminated pre-polymer, among other possibilities) is initially applied to a substrate 100 (e.g., as a solution or suspension), where it reacts with functional groups (e.g., hydroxyl groups, among other possibilities) on the substrate surface, thereby forming bonds (e.g., urethane bonds, among other possibilities). The reactive solution or suspension may then be dried at elevated temperature in dry air or nitrogen, forming a reactive surface. Subsequently, a hydrophilic polymer species 120 (e.g., hydroxyl terminated polyacrylic acid, among other possibilities) and a functional species 130 (e.g., silver nanoparticles with hydroxyl functional groups, among other possibilities) are then applied to the reactive surface (e.g., as a solution or suspension), and dried at elevated temperature. In the embodiment shown, hydroxyl functional groups in the hydrophilic polymer species 120 and the functional species 130 react with unreacted isocyanate to form urethane bonds. The result is a covalently bonded multifunctional coating that provides lubricity and an additional functional effect (e.g., an antibacterial function, among numerous other possible functions such as those described above).

As another example, at least one hydrophilic polymer species may be applied to the first reactive priming layer as a second coat, followed by at least one functional species as a third coat. If it is desired to covalently react functional species in the third coat, then the amount hydrophilic polymer species in the second coat should be insufficient to react with all of the coupling species in the first layer (i.e., the amount should be insufficient too exhaust the coupling species). As yet another example, at least one functional species may be applied to the first reactive priming as a second coat, followed by at least one hydrophilic polymer species as a third coat. If it is desired to covalently react the hydrophilic polymer species in the third coat, then the amount of the functional species in the second coat should be insufficient to react with all of the coupling species in the first layer (i.e., the amount should be insufficient too exhaust the coupling species).

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. A medical device comprising a substrate material and a lubricious coating disposed on said substrate, said coating comprising (a) a hydrophilic polymer species, (b) a functional species and (c) a coupling species.

2. The medical device of claim 1, wherein the coupling species is a polyfunctional isocyanate.

3. The medical device of claim 2, wherein the polyfunctional isocyanate is selected from a diisocyanate and an isocyanate terminated pre-polymer.

4. The medical device of claim 1, wherein the hydrophilic polymer species comprises hydroxyl groups, amine groups, thiol groups or a combination thereof.

5. The medical device of claim 1, wherein the hydrophilic polymer species is a homopolymer or copolymer that comprises one or more monomers selected from acrylic acid, acrylamide, vinyl alcohol, ethylene imine, allyl amine, hydroxyethylmethacrylate, saccharides and amino acids.

6. The medical device of claim 1, wherein the hydrophilic polymer species is terminated with hydroxyl groups, carboxyl groups, amine groups, thiol groups or a combination thereof.

7. The medical device of claim 1, wherein the substrate material comprises hydroxyl groups, carboxyl groups, amine groups, thiol groups or a combination thereof.

8. The medical device of claim 1, wherein the functional species provides a therapeutic agent delivery function.

9. The medical device of claim 8, wherein the therapeutic agent is selected from an antimicrobial agent, an anti-inflammatory agent, an analgesic agent and a gene vector.

10. The medical device of claim 1, wherein the functional species provides a diagnostic function.

11. The medical device of claim 10, wherein the functional species detects an analyte selected from a disease marker, a sugar, a protein, hemoglobin Al c, insulin, a liver enzyme, and creatinine

12. The medical device of claim 10, wherein the functional species is selected from ultrasound echogenic particles and near-infrared contrast agents.

13. The medical device of claim 1, wherein the functional species promotes or inhibits adhesion of cells or non-cellular chemical species.

14. The medical device of claim 13, wherein the functional species provides a cell-anchoring function or an anti-fouling function.

15. The medical device of claim 1, wherein the functional species is in the form of a nanoparticle.

16. The medical device of claim 15, wherein the nanoparticle is selected form a silver nanoparticle, a gold nanoshell, a carbon nanotube, a dye-containing nanoparticle, and a therapeutic-agent-releasing nanoparticle.

17. The medical device of claim 1, wherein the functional species comprises hydroxyl groups, carboxyl groups, amine groups, thiol groups or a combination thereof.

18. A method of forming a medical device coating comprising (a) applying a first coating comprising a coupling species to a substrate material and (b) applying to the first coating one or more additional coatings that comprise a hydrophilic polymer species and a functional species.

19. The method of claim 18, wherein the coupling species in a polyfunctional isocyanate.

20. The method of claim 18, wherein the hydrophilic polymer species comprises hydroxyl groups, carboxyl groups, amine groups, thiol groups or a combination thereof, wherein the functional species comprises hydroxyl groups, carboxyl groups, amine groups, thiol groups or a combination thereof, and wherein the substrate material comprises hydroxyl groups, carboxyl groups, amine groups, thiol groups or a combination thereof.
